(19) United States
(12) Patent Application Publication
(10) Pub. No.: US 2007/0232578 A1
(43) Pub. Date: Oct. 4, 2007

(54) Crystalline forms of ciclesonide

(76) Inventors: Pierluigi Rossetto, Balerna (CH); Peter Lindsay MacDonald, Gentilino (CH); Sigalit Levi, Modi'in (IL); Judith Aronhime, Rehovot (IL)

Correspondence Address: KENYON & KENYON LLP, ONE BROADWAY, NEW YORK, NY, 10004, US

(21) Appl. No.: 11/704,057

(22) Filed: Feb. 7, 2007

Related U.S. Application Data
(60) Provisional application No. 60/773,841, filed on Feb. 15, 2006.

(51) Int. Cl. A61K 31/58; C07J 17/00

(52) U.S. Cl. 514/174; 540/100

(57) ABSTRACT

The invention encompasses a crystalline form of ciclesonide characterized by an XRD pattern having peaks at about 11.0, 14.8, 15.7, 16.5, and 22.8 degrees two-theta±0.2 degrees two-theta, methods of its preparation and pharmaceutical compositions thereof.

### RELATED APPLICATIONS

The application claims the benefit of U.S. provisional application Nos. 60/771,654 filed on Feb. 8, 2006 and 60/773,841 filed on Feb. 15, 2006 hereby incorporated by reference.

### FIELD OF THE INVENTION

The present invention encompasses a new crystalline form of ciclesonide and methods for the preparation thereof. The invention also encompasses pharmaceutical compositions comprising the new crystalline form of ciclesonide and to processes for preparation thereof.

### BACKGROUND OF THE INVENTION

Pregna-1,4-diene-3,20-dione,16,17-[[(R)-cyclohexylmethylene]bis(oxy)]-11-hydroxy-21-(2-methyl-1-oxopropoxy)-(11β,16α)-(9CI), ciclesonide, of the formula: 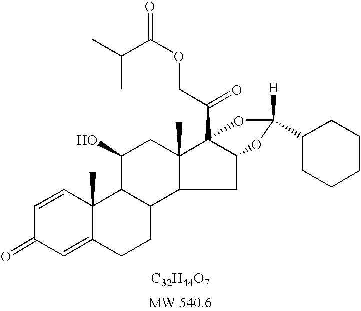
is a non-halogenated glucocorticoid with high local anti-inflammatory properties, that is inhaled in the treatment of asthma. Ciclesonide is an ester prodrug essentially devoid of oral bioavailability, which is activated upon cleavage by endogenous esterases.

The preparation of ciclesonide is disclosed in EP patent No. 929566, PCT publication WO 2004/085460, and U.S. Pat. No. 5,482,934.

Polymorphism, the occurrence of different crystal forms, is a property of some molecules and molecular complexes. A single molecule, like ciclesonide, may give rise to a variety of crystalline forms having distinct crystal structures and physical properties like melting point, x-ray diffraction pattern, infrared absorption fingerprint, and solid state NMR spectrum. One crystalline form may give rise to thermal behavior different from that of another crystalline form. Thermal behavior can be measured in the laboratory by such techniques as capillary melting point, thermogravimetric analysis (“TGA”), and differential scanning calorimetry (“DSC”), which have been used to characterize crystal forms.

The difference in the physical properties of different crystalline forms results from the orientation and intermolecular interactions of adjacent molecules or complexes in the bulk solid. Accordingly, polymorphs are distinct crystalline forms sharing the same molecular formula yet having distinct advantageous physical properties as compared to other crystalline forms of the same compound or complex.

One of the most important physical properties of pharmaceutical compounds is their solubility in aqueous solution, particularly their solubility in the gastric juices of a patient. For example, where absorption through the gastrointestinal tract is slow, it is often desirable for a drug to dissolve slowly so that it does not accumulate in a deleterious environment. This is particularly true when the drug is unstable to conditions in the patient's stomach or intestine. Different crystalline forms or polymorphs of the same pharmaceutical compound can (and reportedly do) have different aqueous solubility.

Also, crystal structure as well as crystal shape (morphology) and size has a significant practical and commercial impact on active substances, especially on formulations for inhalation. The ideal particles for inhalation delivery are spherical of low density and small in size having a narrow size distribution. In principle, size can be controlled by direct crystallization or by micronization. The micronization process may lead to partial crystal form transformation or, in some cases, to the production of amorphous areas. These amorphous areas are more reactive and increase the instability of the active substances because the areas can recrystallize and cause particle bridging. Particle bridging, in turn can increase the particle size. Thus, it is important to produce particles of small size by the crystallization process to avoid particle enlargement when using the micronization process and to ensure that no amorphous form is found in the active substance.

The discovery of new polymorphic forms of a pharmaceutically useful compound provides a new opportunity to improve the performance characteristics of a pharmaceutical formulation. It also enlarges the repertoire of materials that a formulation scientist has available for designing a pharmaceutical dosage form of a drug such as a targeted release profile or other desired characteristic. Because of limited options, there is a need in the art for novel polymorphic forms of ciclesonide, such as those present below.

### SUMMARY OF THE INVENTION

One embodiment of the invention encompasses a crystalline form of ciclesonide characterized by an XRD pattern having peaks at about 11.0, 14.8, 15.7, 16.5, and 22.8 degrees two-theta±0.2 degrees two-theta.

Another embodiment of the invention encompasses a pharmaceutical composition comprising a therapeutically effective amount of crystalline form of ciclesonide characterized by an XRD pattern having peaks at about 11.0, 14.8, 15.7, 16.5, and 22.8 degrees two-theta, ±0.2 degrees two-theta, and at least one pharmaceutically acceptable excipient.

Yet another embodiment of the invention encompasses a process for preparing pharmaceutical compositions of a crystalline form of ciclesonide characterized by an XRD pattern having peaks at about 11.0, 14.8, 15.7, 16.5, and 22.8 degrees two-theta, ±0.2 degrees two-theta, comprising mixing a therapeutically effective amount of a crystalline form of ciclesonide characterized by an XRD pattern having peaks at about 11.0, 14.8, 15.7, 16.5, and 22.8 degrees two-theta, ±0.2 degrees two-theta with at least one pharmaceutically acceptable excipient.

One embodiment of the invention encompasses the use of a crystalline form of ciclesonide characterized by an XRD pattern having peaks at about 11.0, 14.8, 15.7, 16.5, and 22.8 degrees two-theta±0.2 degrees two-theta for the manufacture of a pharmaceutical composition.

### BRIEF DESCRIPTION OF THE FIGURES

The patent or application file contains at least one drawing executed in color. Copies of this patent or patent application publication with color drawings will be provided by the Office upon request and payment of the necessary fee.

FIG. 1 illustrates a powder X-ray diffraction pattern of the crystalline form of ciclesonide obtained by repeating Example 1 of EP patent No. 929566.

FIG. 2 illustrates a powder X-ray diffraction pattern of the crystalline form of ciclesonide of the invention.

FIG. 3 illustrates a photomicrograph of the crystalline form of ciclesonide obtained by repeating Example 1 of EP patent No. 929566.

FIG. 4 illustrates a photomicrograph of the crystalline form of ciclesonide of the invention.

### DETAILED DESCRIPTION OF THE INVENTION

The invention encompasses a new crystalline form of ciclesonide, which is a solvated form of ciclesonide. Purification of ciclesonide via the solvated form providing substantially pure ciclesonide.

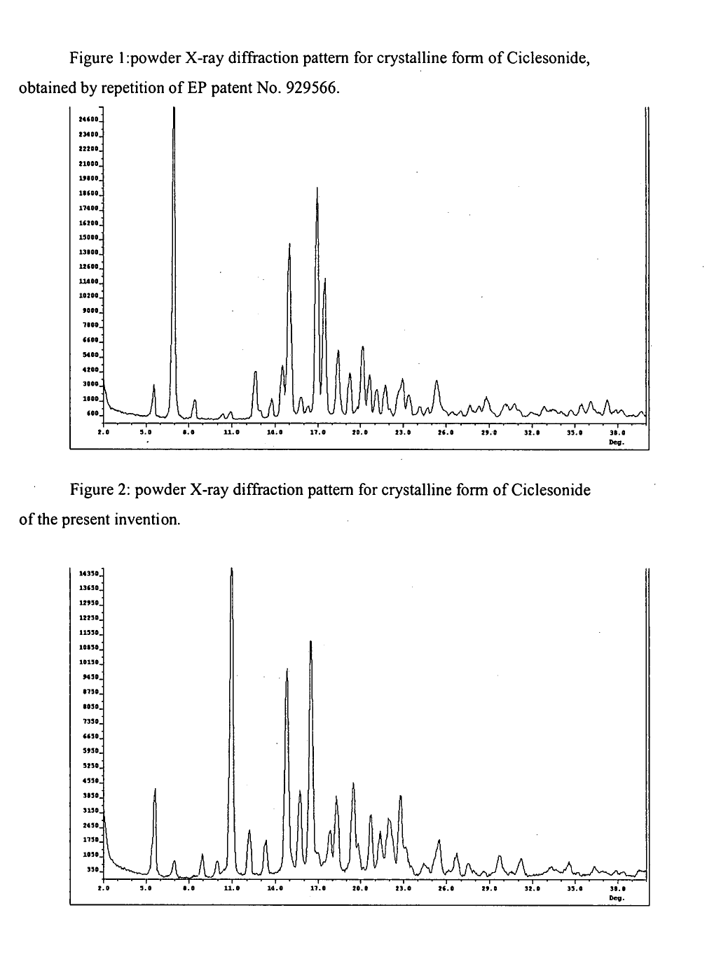

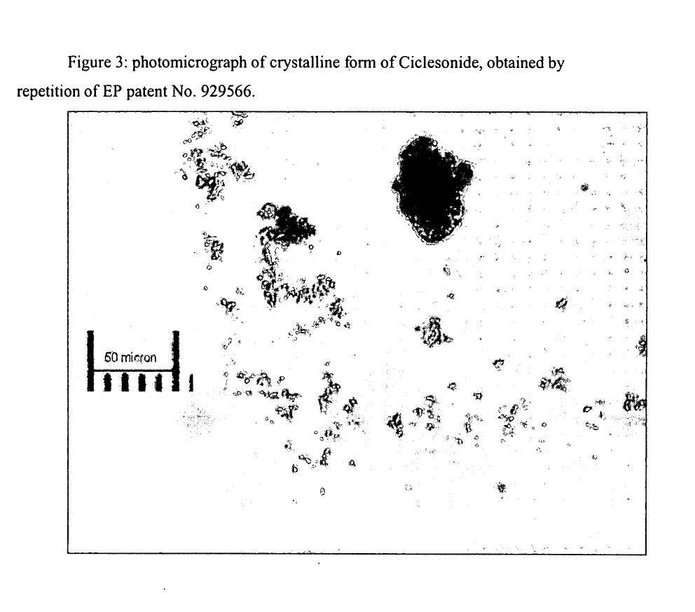

The invention encompasses a novel crystalline form of ciclesonide characterized by an XRD pattern having peaks at about 11.0, 14.8, 15.7, 16.5, and 22.8 degrees two-theta±0.2 degrees two-theta. The crystalline form may be further characterized XRD pattern having peaks at about 5.6, 18.3, 19.5, 20.7 and 22.0 degrees two-theta, ±0.2 degrees two-theta. The crystalline form may also be characterized by an XRD pattern substantially depicted in FIG. 2. The crystalline form may also be characterized by a weight loss of about 10% by weight, as measured at a temperature from about 25° C. to about 130° C. using thermal gravimetric analysis (“TGA”). The crystalline form of ciclesonide of the invention may be further characterized by a melting temperature of about 207° C. The crystalline form may be a solvate. Preferably, the crystalline form is a solvate of tert-butanol in a ratio of 1:1 ciclesonide:tert-butanol (about 12% tert-butanol by weight). The crystalline form can be characterized by any other method known to a skilled artisan, such as solid state NMR and FTIR.

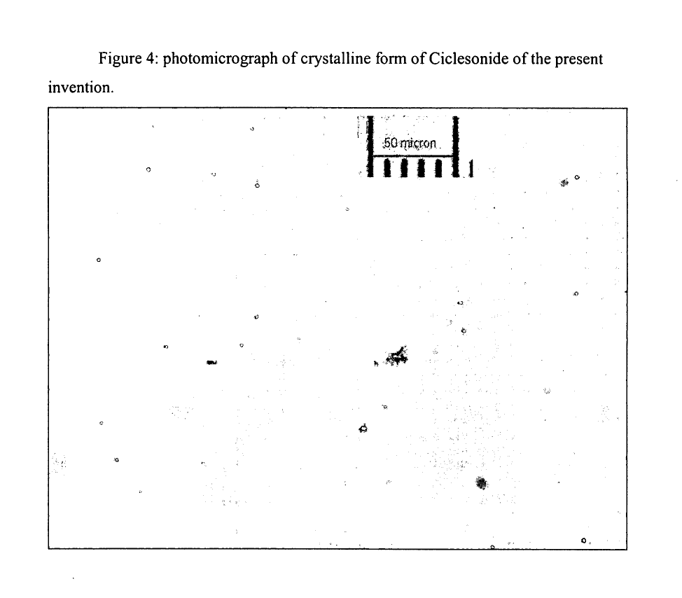

Preferably, the above crystalline form of ciclesonide exists in a morphology of spherical and irregular shape. Typically, as used herein the term “irregular” refers to a crystal that has a morphology which is not spherical or plate shape. The crystalline form may also be substantially identified by a photomicrograph depicted in FIG. 4. This photomicrograph shows that some of the crystals have a spherical shape morphology and some have an irregular morphology. The crystalline form of ciclesonide can have morphology suitable for inhalation delivery.

The invention encompasses a crystalline form of ciclesonide characterized by an XRD pattern having peaks at about 11.0, 14.8, 15.7, 16.5, and 22.8 degrees two-theta, ±0.2 degrees two-theta, and having no more than about 5% by weight of other forms of ciclesonide as determined by XRD. Preferably, the crystalline ciclesonide has no more than 2% by weight of other forms of ciclesonide as determined by XRD. The above form of ciclesonide has not more than about 5% by weight of the crystalline form of ciclesonide characterized by a PXRD pattern having peaks at 7.0, 15.0, 17.0, 17.5, and 18.4 degrees two-theta and additional peaks at 5.6, 12.7, 14.5, 19.3, and 20.2 degrees two-theta. Preferably, the above form of ciclesonide has not more than about 2% by weight of the crystalline form of ciclesonide characterized by a PXRD pattern having peaks at 7.0, 15.0, 17.0, 17.5, and 18.4 degrees two-theta and additional peaks at 5.6, 12.7, 14.5, 19.3, and 20.2 degrees two-theta.

The invention encompasses a process for the preparation of the crystalline form of ciclesonide characterized by an XRD pattern having peaks at about 11.0, 14.8, 15.7, 16.5, and 22.8 degrees two-theta, ±0.2 degrees two-theta, comprising crystallization from t-butanol.

The process for making a crystalline form of ciclesonide characterized by an XRD pattern having peaks at about 11.0, 14.8, 15.7, 16.5, and 22.8 degrees two-theta, ±0.2 degrees two-theta, comprises dissolving ciclesonide in tert-butanol at a temperature of about 50° C. to about 70° C. to form a solution, cooling the solution to a temperature of about 25° C. to about 35° C. to induce ciclesonide crystallization, and isolating crystalline ciclesonide.

The ciclesonide used as the starting material may be made by the methods described in the prior art, such as PCT publication Nos. WO 98/09982 and WO 2004/085460, and U.S. Pat. No. 5,482,934, hereby incorporated by reference. Preferably, the ciclesonide has a purity of at least 99% area by HPLC. For example, the ciclesonide can be obtained by crystallization from a non-hydroxylic organic solvent selected from a group consisting of C₆₋₈ linear or branched aliphatic hydrocarbon, C₂₋₅ ether, and mixtures thereof, as described in U.S. application Ser. No. 11/592,475, filed on Nov. 2, 2006 and hereby incorporated by reference.

One method for making the starting ciclesonide for the invention encompasses crystallizing ciclesonide from a water-immiscible organic solvent. This process comprises dissolving ciclesonide in at least one water-immiscible organic solvent to form a solution; crystallizing ciclesonide from the solution; and recovering the crystallized ciclesonide. After the first crystallization, a solid is obtained which can be crystallized again to further increase its epimeric purity. Preferably the water-immiscible organic solvent is a non-hydroxylic organic solvent.

The starting ciclesonide can be made using methods known in the art, such as the methods disclosed in U.S. Pat. Nos. 2,990,401; 3,929,768; 4,695,625, 4,925,933; 5,482,934; and 5,728,826, and disclosed in PCT publications WO 98/09982 and WO 2004/085460, hereby incorporated by reference. Preferably, the starting ciclesonide contains no more than about 15% of the 22S-epimer as determined by HPLC. More preferably, the starting ciclesonide contains no more than about 12% of the 22S-epimer as determined by HPLC.

Water-immiscible organic solvents include non-hydroxylic organic solvent. The non-hydroxylic organic solvents are organic solvents that lack a hydroxyl group in the chemical compound. Typically, the non-hydroxylic organic solvent includes C₁-C₁₂ straight, branched or cyclic alkanes and C₂ to C₁₂ straight, branched or cyclic ethers. Preferably, the C₁-C₁₂ straight, branched or cyclic alkanes are C₆-C₁₂ straight, branched or cyclic alkanes, and more preferably, C₆-C₈ straight, branched or cyclic alkanes. Preferably, the C₂ to C₁₂ straight, branched or cyclic ethers are C₅ to C₁₂ straight, branched or cyclic ethers, and more preferably, C₅ to C₆ straight, branched or cyclic ethers. Specific examples of C₁-C₁₂ straight, branched or cyclic alkanes include heptane, hexane, and isooctane. Specific examples of C₂ to C₁₂ straight, branched or cyclic ethers include tert-butyl methyl ether, and diisopropyl ether. Preferably, the non-hydroxylic organic solvent is isooctane.

The crystallization process may further employ a second organic solvent, wherein the term second organic solvent refers to an organic solvent that has a boiling point lower than the non-hydroxylic organic solvent. The lower-boiling organic solvent can be any solvent that can dissolve the starting mixture of ciclesonide 22R/22S epimers. Preferably, the lower-boiling organic solvent includes C₁ to C₈ alcohols, C₂ to C₈ ketones, and C₁₋₆ aliphatic halocarbons. Preferably, the C₁ to C₈ alcohols is C₁ to C₅ alcohols, and more preferably, C₂ to C₄ alcohols. Preferably, the C₂ to C₈ ketones are C₂ to C₅ ketones, and more preferably, C₂ to C₃ ketones. Preferably, the C₁₋₆ aliphatic halocarbons are C₁₋₄ aliphatic halocarbons, and more preferably, C₁₋₂ aliphatic halocarbons. Specific examples of C₁ to C₈ alcohols include methanol, ethanol, and tert-butanol. Specific examples of C₂ to C₈ ketones include acetone. Specific examples of C₁₋₆ aliphatic halocarbons include dichloromethane. More preferably, the lower-boiling organic solvent is either acetone or dichloromethane, and most preferably, dichloromethane. The co-solvent is preferably technical grade, i.e. containing less than about 2% water by weight, preferably, less than 1% of water, and more preferably, less than 0.5% by weight.

Typically, when a mixture of a water-immiscible organic solvent and a lower-boiling organic solvent is used, the ratio of the solvents is 20:1 by weight, respectively. Preferably, the ratio is 10:1, and more preferably, the ratio is 5:1 by weight. Optionally, the lower-boiling organic solvent may be removed by evaporation prior to inducing precipitation of the crystalline ciclesonide.

Preferably, the crystallization is performed by dissolving the starting ciclesonide in the water-immiscible organic solvent at a temperature ranging from ambient temperature to about the boiling point of the water-immiscible organic solvent to form a solution, concentrating the solution to obtain a suspension, and cooling the suspension to precipitation of solid ciclesonide. In the embodiment wherein a second lower-boiling organic solvent is used, the process preferably comprises dissolving the starting ciclesonide in the second lower-boiling organic solvent at a temperature ranging from ambient temperature to the boiling point of the second lower-boiling organic solvent, and adding the water-immiscible organic solvent. The mixture may then be concentrated to remove most or all of the second lower-boiling organic solvent, which removal typically results in a suspension.

Typically, the suspension obtained either with or without the second solvent, is cooled to a temperature of about 80° C. to about 10° C. Preferably, the concentrating step is performed by removing the water-immiscible organic solvent by distillation. The precipitate may be separated or recovered using methods commonly known to the skilled artisan. For example, the crystalline ciclesonide may be recovered by filtration. Optionally, the recovered crystalline ciclesonide is washed and dried.

In the process of the present invention, the temperature for dissolving ciclesonide should be sufficient to dissolve ciclesonide in tert-butanol. Preferably, the temperature for dissolving ciclesonide in tert-butanol is about 55° C. to about 65° C., and more preferably, the temperature is about 60° C. Naturally, the cooling temperature should be lower than the dissolving temperature and sufficient to induce the crystallization of ciclesonide. Preferably, the solution is cooled at a temperature of about 30° C. to about 25° C., and more preferably, the temperature is about 27° C. to about 26° C. Optionally, the solution can be stirred during the cooling step.

Any method known in the art can be used to isolate the crystalline ciclesonide. For example, the crystalline form of ciclesonide may be isolated by filtration of the precipitate. Filtration can be carried out by methods including, but not limited to, suction followed by washing of the precipitate. The precipitate can be washed with 2,2,4-trimethylpentane and subsequently dried for about 8 hours in a vacuum oven. Preferably, the vacuum is about 5 to 10 mm Hg. Preferably, the drying step is carried out at a temperature of about 80° C.

The crystalline form of ciclesonide obtained by the crystallization process described above typically contains no more than about 5% by weight of other crystalline forms of ciclesonide as determined by XRD. Preferably, the crystalline form of ciclesonide has no more than 2% by weight of other crystalline forms of ciclesonide as determined by XRD.

The invention also encompasses a process of increasing the R/S epimeric ratio of ciclesonide comprising dissolving ciclesonide having a first R/S epimeric ratio with a sufficient amount of acetone to form a solution and heating the solution to reflux; adding isooctane to the heated solution and heating the solution to about 90° C.; cooling the suspension to about 70° C. for about 30 minutes; allowing the solution to cool to about 25° C. to about 28° C.; then to and collecting the crystalline ciclesonide, wherein the crystalline ciclesonide has a second R/S epimeric ratio and the second R/S epimeric ratio is greater than the first R/S epimeric ratio. Optionally, the process further comprises drying the collected crystalline ciclesonide at a temperature of about 80° C. under vacuum.

Typically, the ratio of acetone to isooctane is about 1:1-40 by volume. Preferably, the ratio of acetone to isooctane is about 1:2-30 by volume, and more preferably, the ratio of acetone to isooctane is about 1:5-20 by volume. Typically, the second R/S epimeric ratio is about 96.5:3.5, preferably about 98:2, and more preferably, 99:1, and most preferably about 99.75:0.25 as determined by HPLC area.

The invention also encompasses pharmaceutical compositions comprising a crystalline form of ciclesonide characterized by an XRD pattern having peaks at about 11.0, 14.8, 15.7, 16.5, and 22.8 degrees two-theta, ±0.2 degrees two-theta, and at least one pharmaceutically acceptable excipient. The invention also encompasses processes for the preparation of pharmaceutical compositions of a crystalline form of ciclesonide characterized by an XRD pattern having peaks at about 11.0, 14.8, 15.7, 16.5, and 22.8 degrees two-theta, ±0.2 degrees two-theta, comprising mixing a therapeutically effective amount of a crystalline form of ciclesonide characterized by an XRD pattern having peaks at about 11.0, 14.8, 15.7, 16.5, and 22.8 degrees two-theta, ±0.2 degrees two-theta with at least one pharmaceutically acceptable excipient.

As used herein unless otherwise defined, the term “therapeutically effective amount” refers to an amount of ciclesonide sufficient to achieve the desired biological effect. Preferably, the amount of ciclesonide is sufficient to act as a bronchodilator when administered to a patient suffering from a respiratory disease, such as asthma. The amount will depend upon the method of use, the age, sex, and condition of the patient, which a skilled artisan can easily determine with little or no experimentation.

The amount of the crystalline form of ciclesonide in a pharmaceutical composition for treating atherosclerosis or hypercholesterolemia should be sufficient to treat or ameliorate atherosclerosis or hypercholesterolemia.

The invention also encompasses the use of a crystalline form of ciclesonide characterized by an XRD pattern having peaks at about 11.0, 14.8, 15.7, 16.5, and 22.8 degrees two-theta, ±0.2 degrees two-theta, for the manufacture of a pharmaceutical composition comprising the same.

Pharmaceutical compositions comprising a crystalline form of ciclesonide characterized by an XRD pattern having peaks at about 11.0, 14.8, 15.7, 16.5, and 22.8 degrees two-theta, ±0.2 degrees two-theta, may be prepared by combining the crystalline form with at least one diluent or excipient, wherein the diluent or excipient includes, but is not limited to, carriers, fillers, bulking agents, binders, wetting agents, disintegrating agents, disintegration inhibitors, absorption accelerators, adsorbing agents, surface active agents, or lubricants. The pharmaceutical composition of the invention should maintain the novel crystalline form of ciclesonide (no solution formulations); however, methods of making pharmaceutical compositions of the invention may include solutions. Additional ingredients, such as dissolving agents, buffer agents, and analgesic agents may be added. If necessary, coloring agents, preservatives, perfumes, seasoning agents, sweetening agents, and other medicines may also be added to the desired preparations. The pharmaceutical compositions may be shaped into various modes for the administration of the pharmaceutical compositions including, but not limited to, tablets, pills, powders, suspensions, emulsions, granules, capsules, suppositories, or injection preparations.

Any excipient commonly known and used widely in the art can be used in the pharmaceutical composition. Carriers used include, but are not limited to, lactose, white sugar, sodium chloride, glucose, urea, starch, calcium carbonate, kaolin, crystalline cellulose, silicic acid, and the like. Binders used include, but are not limited to, water, ethanol, propanol, simple syrup, glucose solutions, starch solutions, gelatin solutions, carboxymethyl cellulose, shelac, methyl cellulose, potassium phosphate, polyvinylpyrrolidone, and the like. Disintegrating agents used include, but are not limited to, dried starch, sodium alginate, agar powder, laminalia powder, sodium hydrogen carbonate, calcium carbonate, fatty acid esters of polyoxyethylene sorbitan, sodium laurylsulfate, monoglyceride of stearic acid, starch, lactose, and the like. Disintegration inhibitors used include; but are not limited to, white sugar, stearin, coconut butter, hydrogenated oils, and the like. Absorption accelerators used include, but are not limited to, quaternary ammonium base, sodium laurylsulfate, and the like. Wetting agents used include, but are not limited to, glycerin, starch, and the like. Adsorbing agents used include, but are not limited to, starch, lactose, kaolin, bentonite, colloidal silicic acid, and the like. Lubricants used include, but are not limited to, purified talc, stearates, boric acid powder, polyethylene glycol, and the like.

Optionally, tablets can be coated with commonly known coating materials such as those coatings used in sugar coated tablets, gelatin film coated tablets, tablets coated with enteric coatings, tablets coated with films, double layered tablets, and multi-layered tablets.

When shaping the pharmaceutical composition into a pill form, any commonly known excipient used in the art to make pills can be used. For example, when making pills carriers include, but are not limited to, lactose, starch, coconut butter, hardened vegetable oils, kaolin, talc, and the like. When making pills, binders used include, but are not limited to, gum arabic powder, tragacanth gum powder, gelatin, ethanol, and the like. Disintegrating agents used include, but are not limited to, agar, laminalia, or the like.

For the purpose of shaping the pharmaceutical composition in the form of suppositories, any commonly known excipient used in the art can be used. For example, the excipients used in suppositories include, but are not limited to, polyethylene glycols, coconut butter, higher alcohols, esters of higher alcohols, gelatin, or semisynthesized glycerides.

When preparing injectable pharmaceutical compositions, solutions and suspensions are sterilized and are preferably made isotonic to blood. Injection preparations may use carriers commonly known in the art, including, but not limited to, water, ethyl alcohol, propylene glycol, ethoxylated isostearyl alcohol, polyoxylated isostearyl alcohol, or fatty acid esters of polyoxyethylene sorbitan. One of ordinary skill in the art can easily determine with little or no experimentation the amount of sodium chloride, glucose, or glycerin necessary to make the injectable preparation isotonic.

The pharmaceutical compositions of the invention may be administered in a variety of methods depending on the age, sex, and symptoms of the patient. For example, tablets, pills, solutions, suspensions, emulsions, granules and capsules may be orally administered. Injection preparations may be administered individually or mixed with injection transfusions such as glucose solutions and amino acid solutions intravenously. If necessary, the injection preparations may be administered intramuscularly, intracutaneously, subcutaneously or intraperitoneally. Suppositories may be administered into the rectum.

Having described the invention with reference to certain preferred embodiments, other embodiments will become apparent to one skilled in the art from consideration of the specification. The invention is further defined by reference to the following examples describing in detail the synthesis of ciclesonide and fractional crystallization thereof. It will be apparent to those skilled in the art that many modifications, both to materials and methods, may be practiced without departing from the scope of the invention.

### EXAMPLES

X-Ray powder diffraction data was obtained by using method known in the art using a SCINTAG powder X-Ray diffractometer model X'TRA equipped with a solid-state detector. Copper radiation of 1.5418 Å was used. A round aluminum sample holder with zero background was used. The scanning parameters included: range: 2-40 degrees two-theta; scan mode: continuous scan; step size: 0.05 deg.; and a rate of 5 deg/min. All peak positions are within±0.2 degrees two theta.

### Comparative Example 1

### Repetition of Example 1 of EP 929566

Ciclesonide (>99% by HPLC) was dissolved in 3.37 parts of absolute ethanol at reflux; and 1.96 parts of water were added to the boiling mixture. The mixture was then allowed to cool to RT with vigorous stirring, and the precipitate was filtered off with suction, washed with 1.58 parts of absolute ethanol/water (2/1 v/v) and dried for 5 h at 50° C. under vacuum, to obtain a crystalline ciclesonide. The crystalline material was analyzed by PXRD showing a pattern having peaks at about 7.0, 15.0, 17.0, 17.5 and 18.4 degrees two-theta±0.2 degrees two-theta, and additional peaks at 5.6, 12.7, 14.5, 19.3 and 20.2 degrees two-theta±0.2 degrees two-theta.

### Example 2

### Preparation of the Crystalline Form of Ciclesonide Characterized by a PXRD Pattern having Peaks at about 11.0, 14.8. 15.7, 16.5, and 22.8 Degrees Two-Theta, ±0.2 degrees two-theta

Ciclesonide (>99% by HPLC) was dissolved at 70° C. in 6.25 parts of tert-butanol and 10.0 parts of water. The solvent was distilled off with vacuum, and 5.0 parts of water were added. The mixture was allowed to cool to R.T. under vigorous stirring, and the precipitate was filtered off with suction, and washed with 5.0 parts of water.

### Example 3

### Preparation of the Crystalline Form of Ciclesonide Characterized by a PXRD Pattern having Peaks at about 11.0, 14.8, 15.7, 16.5, and 22.8 degrees two-theta, ±0.2 Degrees Two-Theta

Ciclesonide (>99% by HPLC) was dissolved in 3.0 parts w/w of tert-butanol at 60° C. The solution was allowed to cool to 26° C. to 27° C. under stirring, and the precipitate was filtered off with suction, washed with 2.0 parts w/w of 2,2,4-trimethylpentane and dried for 8 h at 80° C. under vacuum. The melting point was 206.9° C. The dried precipitate was analyzed by XRD showing a crystalline ciclesonide characterized by an XRD pattern having peaks at about 11.0, 14.8, 15.7, 16.5, and 22.8 degrees two-theta, ±0.2 degrees two-theta and having no more than 5% by weight of crystalline form of ciclesonide having XRD showing a pattern having peaks at about 7.0, 15.0, 17.0, 17.5 and 18.4 degrees two-theta±0.2 degrees two-theta, and additional peaks at 5.6, 12.7, 14.5, 19.3 and 20.2 degrees two-theta±0.2 degrees two-theta.

### Example 4

### Preparation of the Starting Ciclesonide

### Example 4a

### Preparation of Ciclesonide

Desonide 21 -isobutyrate (70 g, 144 mmol) were added in portions at about −20° C. to 73% hydrofluoric acid (350 g), and to the resulting solution was added, during ca. 5 minutes, 18.4 grams (164 mmol) of cyclohexanecarboxaldehyde. The reaction mixture was held at −10° C. to −15° C. for 1 hour, then at ca. −30° C. for 2 hours, and then poured into an ice-cold mixture of 26% ammonium hydroxide solution (87.5 grams) and water (2625 grams). The suspension was stirred for 1 hour, then the precipitate was collected at the filter and rinsed with water.

In order to ensure the absence of acidity, the humid precipitate was distributed between 1000 grams of dichloromethane and 1000 grams of water (adjusted to pH 8 with ammonium hydroxide solution). The organic phase was concentrated at atmospheric pressure to an oily residue (crude product) having an R/S epimer ratio of about 90/10.

### Example 4b

### First Crystallization

The oil of example 4a was dissolved in 280 grams of acetone at reflux and the solution diluted, whilst maintaining under reflux, with 1400 grams of isooctane and concentrated at atmospheric pressure until the temperature of the suspension reached 90° C. The suspension was cooled under agitation to about 70° C. during 30 minutes, and the precipitate was collected at the filter and rinsed with isooctane. The crystals were dried at 80° C. under vacuum to give 64 grams of ciclesonide with an R/S epimer ratio 96.5/3.5.

### Example 4c

### Second Crystallization

The product of Example 4b was recrystallized in the same manner as disclosed in Example 4b using 96 grams of acetone and 1400 grams of isooctane to give 56.8 grams of ciclesonide with an R/S epimer ratio 98.3/1.7.

### Example 4d

### Third Crystallization

The product of Example 4c was recrystallized in the same-manner as disclosed in Example 4b using 85 grams of acetone and 1400 grams of isooctane to give 50.5 grams of ciclesonide with an R/S epimer ratio 99.3/0.7.

### Example 4e

### Fourth Crystallization

The product of Example 4d was recrystallized in the same manner as disclosed in Example 4b using 76 grams of acetone and 1400 grams of isooctane to give 45.9 grams of ciclesonide with an R/S epimer ratio 99.75/0.25.

What is claimed is:

1. A crystalline form of ciclesonide characterized by an XRD pattern having peaks at about 11.0, 14.8, 15.7, 16.5, and 22.8 degrees two-theta±0.2 degrees two-theta.

2. The crystalline form of ciclesonide according to claim 1 further characterized by an XRD pattern having peaks at about 5.6, 18.3, 19.5, 20.7 and 22.0 degrees two-theta, ±0.2 degrees two-theta.

3. The crystalline form of ciclesonide according to claim 2, wherein the crystalline form is further characterized by a PXRD pattern depicted in FIG. 2.

4. The crystalline form of ciclesonide according to claim 1, wherein the crystalline form has a weight loss of about 10% by weight as measured by thermal gravimetric analysis at a temperature from about 25° C. to about 130° C.

5. The crystalline form of ciclesonide according to claim 1, wherein the crystalline form of ciclesonide has a melting point at about 207° C.

6. The crystalline form of ciclesonide according to claim 1, wherein the crystalline form is a solvate of tert-butanol.

7. The crystalline form of ciclesonide according to claim 1, wherein the crystalline form has a morphology of a spherical and irregular shape.

8. The crystalline form of ciclesonide according to claim 7, wherein the crystalline form is further characterized by a photomicrograph depicted in FIG. 4.

9. The crystalline form of ciclesonide according to claim 1, wherein the crystalline form of ciclesonide has no more than about 5% by weight of other forms of ciclesonide as determined by PXRD.

10. A process for making a crystalline form of claim 1, comprising crystallizing ciclesonide from tert-butanol.

11. The process according to claim 10, wherein the crystallization comprises:
dissolving ciclesonide in a sufficient amount of tert-butanol at a temperature of about 50° C. to about 70° C. to form a solution; and
cooling the solution to a temperature of about 25° C. to about 35° C. to induce ciclesonide crystallization.

12. The process for making a crystalline form of ciclesonide according to claim 11, wherein the temperature of the dissolving step is about 55° C. to about 65° C.

13. The process for making a crystalline form of ciclesonide according to claim 11 further comprising isolating the crystalline form.

14. A pharmaceutical composition comprising a therapeutically effective amount of crystalline form of claim 1 and at least one pharmaceutically acceptable excipient.

15. A process for preparing pharmaceutical compositions of the crystalline form of claim 1 comprising mixing a therapeutically effective amount of a crystalline form of claim 1 with at least one pharmaceutically acceptable excipient.

16. A method of treating asthma comprising administering a therapeutically effective amount of a crystalline form of ciclesonide characterized by an XRD pattern having peaks at about 11.0, 14.8, 15.7, 16.5, and 22.8 degrees two-theta, ±0.2 degrees two-theta to a patient in need of treatment thereof.

17. A method of treating atherosclerosis or hyhpercholesterolemia comprising administering a therapeutically effective amount of a crystalline form of ciclesonide characterized by an XRD pattern having peaks at about 11.0, 14.8, 15.7, 16.5, and 22.8 degrees two-theta, ±0.2 degrees two-theta to a patient in need of treatment thereof.
